(19) United States
(12) Patent Application Publication
(10) Pub. No.: US 2018/0280265 A1
(43) Pub. Date: Oct. 4, 2018

(54) COATING FOR ANIMALS TO MANAGE SOLAR RADIATION

(71) Applicant: Kansas State University Research Foundation, Manhattan, KS (US)

(72) Inventors: Steven J. Bartle, Manhattan, KS (US); Daniel U. Thomson, Riley, KS (US); Christopher D. Reinhardt, Manhattan, KS (US)

(21) Appl. No.: 15/570,655

(22) PCT Filed: Apr. 29, 2016
(86) PCT No.: PCT/US16/30104

§ 371 Date: Oct. 30, 2017

Related U.S. Application Data
(60) Provisional application No. 62/154,841, filed on Apr. 30, 2015.

(51) Int. Cl. A61K 8/29; A61Q 17/04; A61K 8/27; A61K 8/19; A01K 13/00

(52) U.S. Cl. CPC A61K 8/29; A61Q 17/04; A61K 8/27; A61K 8/19; A01K 13/003; A61K 2800/43; A61K 2800/242; A61K 2800/244; A01K 13/006

(57) ABSTRACT

Coatings aimed at improving an animal's health and well-being are provided. In particular, the coatings can help the animal manage its exposure to solar radiation, either through increased absorption of solar radiation in cold environmental conditions, or through increased reflectance of solar radiation in hot environmental conditions. In addition, or alternatively, the coatings can be used to deliver fragrances to mask the presence of bullers or protect the animal from infections or insects.

### RELATED APPLICATION

This application claims the benefit of U.S. Provisional Patent Application No. 62/154,841, filed Apr. 30, 2015, which is incorporated by reference herein in its entirety.

### BACKGROUND OF THE INVENTION

### Field of the Invention

The present invention is generally directed toward animal coatings that improve the animal's health and well-being. In certain embodiments, the coatings can help manage solar radiation. Management of solar radiation can include absorbing infrared radiation so as to warm then animal when ambient temperatures are cold, or reflecting infrared radiation so as to help the animal maintain a lower body temperature when the ambient temperatures are hot. In other embodiments, the coatings can be used to deliver fragrances to mask the presence of bullers, or protect the animal from infections or insects.

### Description of the Prior Art

Heat stress causes multiple problems for the livestock industries. Heat stress happens when heat gain exceeds heat loss. High ambient temperatures and elevated humidity reduce the animal's ability to dissipate heat. When these conditions are combined with high heat absorption from solar radiation, heat stroke and death can occur. In 2014 it was reported in seven years since 1995 over 5,000 feedlot cattle have been documented to have died of heat stress and it was estimated that non-death financial losses are 5 to 10 times greater than death losses. Mader, T. L., 2014. Bill E. Kunkle interdisciplinary beef symposium: Animal welfare concerns for cattle exposed to adverse environmental conditions. J. Anim. Sci. 92:5319-5324. The economic losses to unabated heat stress in the United States were estimated in 2003 at $2.4 billion with roughly 75% of the losses occurring in the dairy and beef industries. In 2003, the price for finished beef cattle was $80 to $85/cwt, in 2014 the prices are $135 to $145/cwt. In addition to financial losses, cattle suffer pain and discomfort during periods of heat stress, especially if they are not able to dissipate accumulated heat at night.

Currently, three major management practices are used to address heat stress problems in the beef industry: 1) Being selective in the type of cattle fed during the summer; light weight cattle, and cattle with some Brahman genetics tolerate the heat better.

However, the demands for a consistent beef supply dictate that many cattle do not fit these criteria; 2) Shades—some feedlots build shades, but the vast majority do not, which is a strong indication that the returns do not support the investment at this time; and 3) Sprinkling with water and evaporative cooling. Sprinkling is expensive in terms of equipment, labor and dollars. Also, water is becoming limited in several areas of the country where cattle are fed.

In cold climates, the opposite problem of heat loss is an issue, causing a decrease in production for food animals as they burn extra calories in an effort to stay warm. In these situations, what is needed is a way to increase heat absorption. These problems are not unique to feedlot and dairy cattle and can be observed in other animals that spend a lot of time outside. There remains a need for an inexpensive, easy to use and effective method of managing solar radiation in animals.

Food animals, especially those harvested from feedlots, can carry a number of contaminants on their hair and hides, such as mud, fecal matter and various pathogens like E. coli. These contaminants can be spread from the animal's hide to carcass during processing of the animal carcass into food. However, no methods are currently available to reduce the risk of contamination from the hide to the carcass of food animals before they reach the slaughter facility. There is a need for a method of reducing the risk of contamination from the animal's hide to the carcass that can be carried out before the animal reaches the slaughter facility.

Buller-Steer Syndrome is a behavior problem where several steers have a sexual attraction to a small number of other steers (termed the buller) in the feedlot pen. The buller is ridden by the other steers resulting in poor performance, reduced weight gains, and injury or death. The Buller-Steer Syndrome ranks as a top health problem in the feedlot industry. To date, no effective methods are available to solve the problem of bullers without removing them from their home pens, or the dominance fighting that happens when animals are co-mingled. Thus, there is a need for an alternative to segregating bullers from the rest of the herd.

Insects are known to cause certain problems in food animals. In addition to being irritating to the animals, insects may also carry and transmit diseases within a herd of animals. There is a need for a method of reducing the negative effects of insects on populations of animals living in close confines.

### SUMMARY OF THE INVENTION

One embodiment of the invention comprises a method of decreasing solar heat radiation and absorption on an animal, said method comprising applying a highly infrared (IR) and solar reflective material to the skin or hide of the animal. In one embodiment of the invention, the highly reflective material is a metal oxide such as titanium dioxide or zinc oxide.

One embodiment of the invention comprises a method of increasing solar heat radiation transfer to an animal by applying a highly infrared and thermally absorptive material to the skin or hide of the animal. In one embodiment of the invention, the highly absorptive material is iron oxide or carbon black.

In one embodiment, the invention provides for products that are specifically formulated to manage solar radiation. Preferably the material being applied to the animal will be non-toxic to the animal, and in the case of food animals, also be approved for use in the food industry. Additionally, the material should not adversely affect the condition of the hide.

In another embodiment, the invention provides for compositions and methods for managing the heat effects of solar radiation on an animal. The compositions generally comprise a formulation that includes a solar reflective material and a material that slowly releases water so as to provide an evaporative cooling effect for the animal to which the composition has been applied. In particular embodiments, in addition to a solar reflective material, the composition also comprises a mixture of water and clays. The water is slowly released from the clay mixture and evaporates on the animal's fur or hide to provide an evaporative cool effect.

In another embodiment, the invention provides for compositions and methods for reducing the contaminants present on the hides of food animals. The compositions can be formulated to directly reduce the concentration of microbes on the hide, and/or reduce the adherence of mud, fecal matter, or other contaminants to the hide. As with the embodiments noted above, the compositions should be non-toxic to the animal, approved for food-grade uses, and not adversely affect the condition of the hide.

In still another embodiment, the invention provides for compositions and methods for addressing Buller-Steer Syndrome and co-mingling stresses brought about by placing of the food animals within a feedlot environment in which the animals are confined to pens. The methods can involve applying a composition to at least a portion, and preferably all, of the animals in the same confined area or pen that includes a specific aroma causing the animals to smell alike. In this manner, the identification of bullers and new animals added to a particular environment can be masked so that the bullers could not be identified and dominance fighting among the animals could be reduced.

In still another embodiment, the invention provides for methods of reducing the effects of biting or stinging insects on the animal through application of a protective composition to the hide of an animal, especially a food animal that directly repels the insects away from the animal and/or confuses the insects so that the animal becomes a less inviting target.

### BRIEF DESCRIPTION OF THE DRAWINGS

FIG. 1 is a photograph showing application of a reflective coating to the backs of cattle; and

FIG. 2 is a chart showing differences in body temperature increase between coated and uncoated cattle.

### DETAILED DESCRIPTION OF THE PREFERRED EMBODIMENT

When light waves hit a substance, there are three major outcomes. The light can be transmitted, absorbed, or bent (refracted). Reflectance is bending light waves so they do not pass through the material. Highly reflective materials are efficient at bending the light waves and redirecting them back to the environment. Reflectance is the result of refractance. Solar heat is generated primarily by the visible and near infrared wavelengths (400 to 1200 nm).

In embodiments of the present invention, it has been discovered that reflecting solar radiation so that heat is directed away from the animal's hide and not transferred from the solar radiation into the body is a way to reduce heat stress in feedlot cattle.

Titanium dioxide and zinc oxide are two examples of highly reflective metal oxides. Importantly, both are approved to be fed to cattle (2014 Official Publication—Association of American Feed Control Officials). Other examples of solar reflective and infrared (IR) reflective materials are well known in the art. Their overall effectiveness depends upon the darkness of the coating color and the amount of direct exposure to sunlight. A range of efficient colors can be used; however, the color white is the most reflective of all colors and is therefore the most preferred, however, not all whites are the same. The percentage concentration of reflective material such as titanium dioxide, its particle size and purity, and the type and quality base it is blended into all contribute to infrared reflectivity. Coating a white surface with white IR reflective materials will still provide an increase in reflectivity and decrease solar radiation.

Conversely, applying pigments with known IR and solar radiation absorption can be used to increase heat on animals in cold climates. Examples of solar spectral radiative properties of conventional and cool pigmented coatings can be found at the Lawrence Berkeley National Pigment Database. http://coolcoolers.lbl.gov/LBNL-Pigment-Database/database.html

Heat stress in animals can result in decreased feed consumption, decreased weight gain, a reduced period of estrus and a resulting decrease in the number of births, as well as suffering and death. Conversely, extreme cold temperatures can also result in decreased weight gain as more calories are used by the animal to stay warm, increased feed costs, suffering and death. Therefore, embodiments of the present invention provide for products that are specifically formulated to manage solar radiation, both reflective and absorptive.

A liquid comprising one or more active ingredients, a non-toxic adhesive to bind the liquid to the hide or hair of the animal, a surfactant/wetting agent or dispersant to make a suspension concentrate and water is provided. The suspension concentrate being a uniform mixture of liquid and a non-soluble solid (IR reflective pigment). Additional ingredients can be included, such as a bitter intake inhibitor to prevent licking of the product and an identifying pigment that is manufacturer specific. In certain embodiments, the product is produced in a concentrated form and shipped to the end user who would dilute the concentrate and/or apply the product with water. The product can be applied using any liquid application system, including brushes, paint balls and rollers. Preferably the product would be applied using common and commercially available spraying equipment.

The present invention is particularly applicable to feedlot and dairy cattle, which typically represent a large number of animals in a confined area and are therefore easy to treat. Other animals that are subject to heat stress could also be treated, including for example, grazing cattle, companion animals and zoo animals.

Tables 1 and 2 provide estimated formulations and additional information about the ingredients. It should be appreciated that the product formulation can be adjusted depending on the concentration of the shipped product, how often it is to be applied, the temperature and other variable conditions. The coating formulation should remain in place on the animal for at least 7 days, and more preferably for at least 10 days, and most preferably about 14 days, before de-adhering from the animal's hide through rubbing, licking, or weather erosion.

TABLE 1
Reflectance product example formulation

| | Content | | | |
|---|---|---|---|---|
| Ingredient | Broad | Intermediate | Narrow | Reflectance/Purpose |

Active ingredients

| | | | | |
|---|---|---|---|---|
| Titanium dioxide | 10 to 50% | 15% to 45% | 20% to 40% | Refraction score = 2.7; |
| | | | | one of the most |
| | | | | reflective substances |
| | | | | available. |
| | | | | Reflects both visible |
| | | | | and near infrared wave |
| | | | | lengths |
| Zinc oxide | 0 to 20% | 7% to 18 | 10% to 15% | Score = 2.0 |
| | | | | Reflects both visible |
| | | | | and near infrared wave |
| | | | | lengths |
| | | | | Absorbs UV |
| | | | | wavelengths that cause |
| | | | | sunburn |
| Calcium | 0 to 20% | 1% to 15% | 5% to 10% | Score = .6 |
| carbonate | | | | |
| Silicon dioxide | 0 to 20% | 1% to 15% | 5% to 10% | Score 1.6 |

Inactive ingredients

| | | | | |
|---|---|---|---|---|
| Surfactant, | 2 to 50% | 10% to 40% | 20% to 30% | Not reflective |
| dispersant, | | | | Used to create a |
| Wetting agent | | | | suspension concentrate |
| Adhesive | 1 to 20% | 2% to 15% | 5% to 10% | Used to cause active |
| | | | | ingredient to adhere to |
| | | | | hair and hide |
| Water | 20 to 80% | 30% to 70% | 40% to 60% | Used as part of the |
| | | | | suspension concentrate |
| Coloring pigment | .01 to 3% | 0.1% to 2% | 0.5% to 1% | — |
| Intake limiter | .01 to 1.0% | 0.1% to 0.75% | 0.25% to 0.5% | Bitter tasting ingredient |
| | | | | to reduce possible |
| | | | | consumption of the |
| | | | | product, especially |
| | | | | before drying |

TABLE 2
Absorbance product example formulation

| | Content | | |
|---|---|---|---|
| Ingredient | Broad | Intermediate | Narrow |

Active ingredients

| | | | |
|---|---|---|---|
| Carbon black | 10 to 50% | 15% to 45% | 20% to 40% |
| Iron oxide | 10 to 50% | 15% to 45% | 20% to 40% |

Inactive ingredients
Same as reflectance product

The solar and IR reflective material of the invention can be applied to the hide or fur of an animal in order to reduce heat stress in the animal. Similarly, the solar and IR absorptive material of the invention can be applied to the hide or fur of an animal in order to increase heat gain in the animal.

A model has been developed to estimate the performance losses due to heat stress for various locations, environmental conditions, and cattle type. The model estimates the effects of coating at 70% the value of full shade for the Dodge City, KS area, a 1,250 lb steer, and over 90 days in a normal summer. The model indicates that coatings should improve dry matter intake by 2.47% (about 0.6 lb) and daily gain by 4.24% (about 0.17 lb). Calculating economic added value 0.17 lb (@ $1.50/1b) minus the cost of 0.6 lb of feed (@$250/ton) results in a daily profit for the feedlot of $0.21 per day per animal. This figure does not include reduced feedlot deaths or improved carcass characteristics, which are often observed in shade studies.

A best case calculation on the economic benefits might be as follows: Of the 4.7 million feedlot cattle marketed per year in Kansas, about one million head will be in the 1,000+lb range at any one time, so the potential profit to the Kansas feedlot industry is 1,000,000 cattle X 90 summer days X $0.21/day=$18.9 million per summer.

The advantages of coatings over other methods to mitigate heat stress are substantial.
Lower costs—The startup or fixed costs are low and comprise a dedicated commercial sprayer, a spray bar, and possibly a specialized short alley for the cattle to walk through. An estimate would be $5,000 to $25,000. All other costs would be variable, and comprise coating product and labor.
Management flexibility—The feedlot manager would have the ability to manage which cattle, which day, and how often to re-apply.
Not subject to windy conditions—Shades and sprinklers can be destroyed or made ineffective by windy conditions.

The effects of solar reflection to reduce heat stress may be supplemented with a coating composition that also provides evaporative cooling characteristics. In certain embodiments, the coating composition can be formulated to comprise water and clays to provide the slow release of water from the coating, which would evaporate resulting in a cooling effect for the animal. Like the coatings discussed above, a reflective material may be used in the coating composition to provide beneficial solar reflection characteristics. Such a composition may be particularly applicable in emergency situations where the animal was suffering severe heat stress and is in danger of dying. In certain embodiments, these reflective and evaporative coatings may have an effective life of between about 2 to about 6 hours before re-application would be necessary. These coating compositions need not be limited for use on feedlot cattle, but can be used on any animal that is suffering heat stress such as hunting dogs, draft animals, and service animals.

The base formulation from Table 1, above, namely the inactive ingredients, can also be used in formulating animal coating compositions. For example, the base formulation can be used to formulate germicidal coating compositions to as to reduce the contaminants on the hides of food animals. These compositions may include various germicidal agents including one or more compounds selected from the group consisting of quaternary ammonium compounds, benzalkonium chloride, cetyl trimethylammonium bromide, cetylpyridinium chloride, and benzethonium chloride, boric acid, chlorhexidene gluconate, iodine, phenolic antiseptics, chelated silver compounds, aliphatic alcohols, peroxides, and aldehydes. Alternatively, the base formulation can be used to formulate coatings that, when applied to the animal's hide, reduce the adherence of various soils, such as mud and fecal matter. Such coatings may include food grade vegetable and petroleum waxes, food-grade polymers and surfactants, silanes, siloxanes, and silicates.

In certain embodiments, the foregoing coating compositions are applied to a substantial portion of the animal hide; however this need not always be the case in order to reap the benefits of the composition. Rather, and particularly in the case of the coatings that prevent adherence of soils, the coating composition need only be applied to those portions of the animal most likely to come into contact with such soils, especially the animal's hooves, legs, hind quarters, and bellies.

The base formulation from Table 1 can also be used to formulate compositions that can be applied to food animals, namely cattle, to address Buller-Steer Syndrome. These compositions may be formulated with various volatile and non-volatile organic compounds to provide a desired therapeutic effect. Exemplary organic compounds include animal and plant-derived oils, extracts of fruits, flowers, seeds, woods, and barks, various resins, terpenes, ambergris, castoreum, civet, hyraceum, honeycomb, and musk. The base formulation is sufficient to adhere the organic compounds to the animal's hide so that the animal gives off an aroma that masks the identity of bullers and animals that are new to the herd.

Methods of treating animals with various aromatic compositions include applying the composition to one or more animals present within the herd, such as those animals occupying a common pen in a feedlot. Preferably, the aromatic composition is applied to a large number of the animals, more preferably all the animals, occupying the common pen. By doing so, all of the animals give off a common scent thereby deterring aggression and masking the scent of bullers present within the herd.

The base formulation from Table 1 can also be used to formulate insect repellant compositions that can be applied to an animal. Such compositions can be formulated with an insect repellant compound such as those selected from the group consisting of diethyltoluamide (DEET), phosmet, permethrin, citronella, essential oils, pyrethrins, zetacypermethrin and piperonyl butoxide. Upon application to the animal's hair or hide, the composition can be effective in repelling nuisance pests such as house flies, stable flies, fire ants, fleas and ticks.

The base formulation from Table 1 may also include materials that improve the water resistance of the compositions. Such materials include acrylic-based polymers and co-polymers, and paraffin/polyethylene wax emulsions. Thickening agents can also be employed in order to adjust the viscosity of the coating to ensure that the coating stays in place on the animal's hide until it has an opportunity to sufficiently dry. In order to further aide adherence, food-grade tackifiers (e.g., Functional Products V-801) and binders (e.g., hydrolyzed casein) may also be employed. Food grade solvents and/or humectants (e.g., triacetin) may also be employed in order to delay loss of moisture from the composition after application to the animal's hide. By delaying evaporation of the moisture from the composition, a further cooling effect can be realized over time.

A number of potential uses for the present invention to adhere various compounds to the hair and hide of animals are summarized in Table 3.

TABLE 3
Conditions to be addressed through adherence of compounds to animal hides

| | Compound | | |
|---|---|---|---|
| Problem/Issue | adhered | Action | Animal |
| Heat stress | Reflective | Reflect solar radiation | Beef and dairy |
| | materials | | cattle, |
| | | | Idle horses |
| | | | Dogs |
| Heat stress | Reflective | Reflect solar radiation | Heat stressed |
| emergency | materials and | Evaporative cooling | animals |
| | cooling compound | | |
| Cold stress | Absorptive | Absorb solar radiation | Beef and dairy |
| | materials | | cattle |
| | | | Idle horses |
| | | | Dogs |
| Food safety | Germicidal/ | Reduce contamination of | Beef and dairy |
| | antimicrobial | the hide prior to slaughter | cattle |
| | compounds | | Swine |
| | | | Poultry |
| “Bullers” | Aroma | Make all animals smell | Feedlot steers |
| Co-mingling | | alike | Any co-mingled |
| | | | animals |
| Insect | Insecticides | Reduce the incidence of | Beef and dairy |
| “repellents” | | biting insects harassing | cattle |
| | | animals | Horses |
| | | | Swine |

### EXAMPLES

The following examples set forth various compositions and methods in accordance with the present invention. It is to be understood, however, that these examples are provided by way of illustration and nothing therein should be taken as a limitation upon the overall scope of the invention.

### Example 1

Table 3 provides an exemplary reflective composition in accordance with the present invention. Table 4 lists additional, optional components that may be added to the composition of Table 3.

TABLE 3

| Material | CAS No. | Weight % |
|---|---|---|
| Acrylic emulsion (BASF Joncryl DFC 3030) | Proprietary | 10-20 |
| Water | 7732-18-5 | 50-75 |
| Titanium Dioxide | 13463-67-7 | 10-30 |
| Cellulose Gum (Ashland CMC 9H4F) | Proprietary | 0.1-2 |

TABLE 4

| Material | CAS No. | Weight % |
|---|---|---|
| Paraffin/polyethylene wax emulsion (Michem | Proprietary | 1-10 |
| Emulsion 36840) | | |
| Tackifier Additive (Functional Products V-801) | Proprietary | 1-10 |
| Acrylic Emulsion (DSM NeoCryl A-1052) | Proprietary | 5-25 |
| Triacetin | 102-76-1 | 1-10 |
| Hydrolyzed Casein | 65072-00-6 | 1-20 |

### Example 2

In this example, experiments were conducted on plastic containers filled with water to determine the effect of various pigments on energy absorption and reflectance. Laboratory water containers (jugs) were painted red, white, or black using Krylon spray paints. The satin white contained titanium dioxide as the primary pigment at a concentration of less than 15% by weight. Note that spray paints contain propellants that make up 50% or more of the formulation. Each container was filled with water until the jug plus water equaled 30 lb. Recording thermometers were suspended in the water. Jugs were place in a feedlot pen for five days, recording started at 6:00 am. Temperature recorded every 15 minutes. There were 3 jugs per color for 9 total jugs. Hourly ambient temperatures were obtained from the weather station at the Manhattan, KS airport. The ambient daytime temperatures varied between about 70° F. to about 98° F. over the course of the trials.

Comparing the temperatures at 4:00 pm, which was near the hottest time of day, showed that temperatures inside the white jugs averaged about 6° F. lower than the red or black jugs (98° vs 104° , P<0.001). Also, it was noted that the temperature increase in the white jugs over ambient was roughly half as high as for the black jugs, suggesting that the heat accumulation was roughly half as great.

### Example 3

In this example, the effects of titanium dioxide paint on wool felt and heat transfer was examined. Two 8″ square pieced of black wool felt were used. One was coated with an artist's paint, which contained titanium dioxide as the primary pigment (Artists paint MSDS's list only toxic materials, and titanium dioxide is not listed. In house paints, titanium dioxide is in the ingredient section as 10-35%). Four recording thermometers were placed under an 8″ square of wool felt, and a heat lamp with infrared heat bulb was placed about 14″ above the felt. The temperature was recorded every 5 minutes for 90 minutes. The process was repeated on two days for each treatment.

The temperatures under the felt began to separate by 15 min, and by 60 minutes the temperature under the coated felt was about 10° F. lower than under the black felt.

### Example 4

Heat stress in feedlot cattle has serious animal welfare and economic implications. Documented feedlot cattle losses have exceeded 5,000 head in seven of the last 20 years, and non-death costs are estimated at 5 to 10 times greater than death. Environmental conditions including ambient temperature, humidity, wind, and solar radiation can affect heat load. Also, 75 to 77% of domestic beef cattle are black colored. The environmental and animal factors are difficult to control, especially in a cost effective manner.

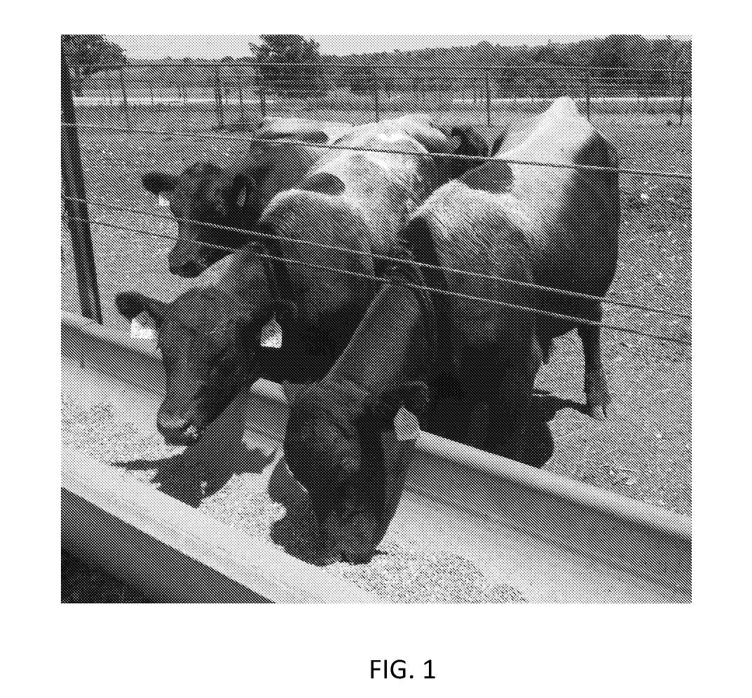

The objective of this experiment was to determine if titanium oxide coatings reflect solar radiation from cattle and mitigate heat stress. Feedlot heifers (n=30, 29 black and 1 red; 591 lb+/−60.8 lb) were used to evaluate a reflective coating containing titanium dioxide. Heifers were randomly assigned to control or coated treatments. The coating was applied to the dorsal midline with an electronic airless sprayer except for the area over the shoulders, which served as a control. See, FIG. 1. Vaginal thermometers attached to blank CIDRs were inserted into six heifers in each treatment to continuously record internal body temperature. Coatings and thermometers were applied from 10:00 to 11:00 am on a day with a high ambient temperature of 101° F. and a temperature humidity index of 87.8. Starting at about 1:00 pm, reflectance surface in the major color zones from the dorsal were measured with a suspended modified digital camera. Hide surface temperature was measured with a suspended infrared thermal imaging sensor; adjacent panels of white, black, and grey provided reference temperatures. A sprinkler was turned on at about 2:30 pm because of severe heat stress in several cattle.

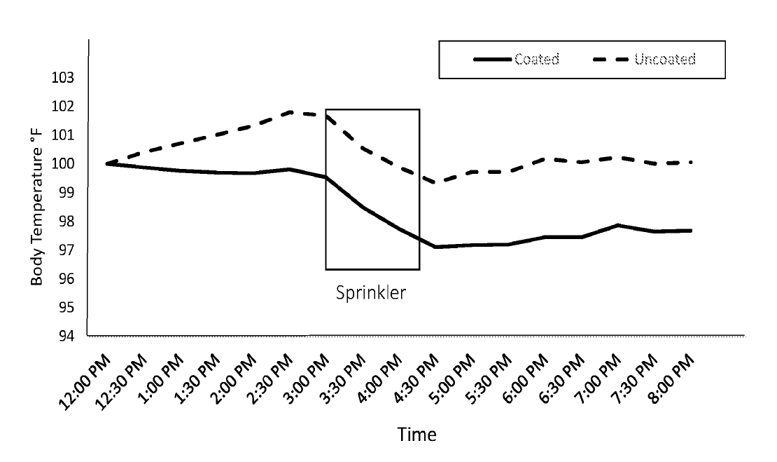

Reflectance and hide surface temperature were compared between two areas of the same animal. Reflectance in the blue, green, and red color zones was found to be 5.7, 8.8, and 10.3 times greater (P<0.001), respectively, for the coated areas (back) than the uncoated (shoulder) areas. Dorsal surface temperature averaged 102.3 and 108.3° F. for coated and uncoated areas, respectively (P<0.001). When a light wave strikes a surface, it can pass through, be reflected away from the surface, or be absorbed and converted to heat. As the amount of light that is reflected increases, the amount absorbed decreases. Reflectance values and hide surface temperatures suggest that solar energy was reflected rather than absorbed. Body temperatures were compared between coated and uncoated animals. Uncoated cattle had a 1.6° F. greater (See, FIG. 2; P<0.01) body temperature increase than coated cattle over a 2 to 3 h exposure to natural solar radiation, which suggests that heat stress was reduced in coated cattle. A reflective coating applied to the dorsal midline of feedlot cattle shows potential to decrease heat absorbed and reduce heat stress.

### Example 5

The objective of this experiment was to determine if a coating containing titanium dioxide would be effective in reflecting solar radiation from the hides of cattle, and thereby mitigate heat stress. A laboratory evaluation was conducted to determine the reflective properties of a titanium dioxide coating cattle hides.

### Materials and Methods

Four hair-on rug hides from Holstein cattle were purchased (Sunland Home Decor, Tucson, Ariz.)). Nine samples about 20 cm on a side were cut from each hide from the black colored shoulder area of the rugs. The nine black samples were randomized and assigned to one of three coating treatments. The treatments were 1) Black, no coating, 2) 50% coverage based on visual appraisal (Mid), and 3) 100% coverage based on visual appraisal (High). Coatings were applied to the black hide samples using a hand sprayer. The samples were allowed to dry for several days before laboratory measurements were conducted. The reflective coating containing titanium dioxide was supplied by a livestock marking paint company.

### Lux Measurements

Light was supplied by a laboratory fiber optic illuminator (Cole Palmer Model 41720) with a halogen EKE 150W bulb. The bulb and the heat it generated were separated from the hide sample by the fiber optic gooseneck. The light pipe lens was placed at a 33° angle about 10 cm from the surface of the hide sample. The lux measurements were taken using a modified Lux meter. The meter was fitted with a cone, which restricted the area of hide reflecting irradiation to the instrument, and with 8 CM legs, which provide a constant height above the hide sample. Before reading for each hide sample the lights were turned off in the windowless laboratory, and the lux meter zeroed. Readings were then taken at low, medium and high settings of the illuminator after a three second adjustment period. Measurements were taken on the 36 hide samples, and then the procedure was repeated two more times so that three readings were taken for each hide sample at each of the three illuminator settings.

### Heat Transfer Measurements

The equipment used to determine heat transfer through the hide sample included the fiber optic illuminator, a thermal camera, a shield which prevented interference from stray light and provide a structure to hold the hide sample, and a timer. The shield comprised a piece of plywood 46 cm on a side, which was painted black. An opening 10 cm on a side was cut in the center of the shield. To measure heat transfer, the hide sample was attached to the shield with the hair side facing the shield and the illuminator. The light pipe lens was placed perpendicular to the hide and 10 cm from the surface of the hide. The thermal camera was placed about 35 cm from the skin side of the sample. At time zero, the skin side temperature was recorded and the illuminator turned on to its maximum power. Skin side temperatures were then recorded every 30 seconds for 10 minutes. Heat transfer was determined on the 36 hide samples, and then the procedure was repeated two more times for a total of three measurements per hide sample.

The relationship between temperature and time were described by Equation 1 and nonlinear regression was used to fit the observed data to this model (Phoenix WinNonlin®; Certara, Cary, N.C.). The data were weighted by the square of the observed values to ensure that higher values were not under-predicted.

$$T = {T}_{0} + \frac{{T}_{max} \times time}{{time}_{50} + time} \qquad Equation 1$$

Where T is the measured temperature, T₀ is the initial temperature, Tₘₐₓ is the maximum increase in temperature, and times( )is the time at which 50% of the maximum increase in temperature has occurred. The temperature at 10 min was calculated by summing the T₀ and Tₘₐₓ values.

### Light Reflectance

The light reflectance of a broad spectrum light source (Ocean Optics HL-2000-FHSA) from untreated and treated skin surfaces, expressed as percent reflectance compared to a polytetrafluoroethylene total reflectance standard (Ocean Optics WS-1), were estimated over a wavelength range of 380 nm to 900 nm using a spectrometer (Ocean Optics Flame-S).

### Statistics

The three replicate measurements of each hide sample were averaged to produce one value for statistical analysis for both lux and heat transfer variables. For the lux variable, the analysis was an ANOVA using the GLIMMIX procedure of SAS; treatment was a fixed effect, and hide and hide X treatment were random effects. Treatments were separated using least squares means and statistical significance was declared at P <0.05.

For the heat transfer data, the time to reach 50% of maximum temperature and the temperature at 10 min were evaluated using GLIMMIX procedure of SAS. Treatment was a fixed effect, and hide and hide X treatment were random effects. Treatments were separated using least squares means and statistical significance was declared at P<0.05.

### Results and Discussion

Lux measurements are shown in Table 5. At the high light level, the Black, Mid and High coating treatments had different (P<0.001) lux values of 1,741, 15,978, and 40,730, respectively, which corresponded to an overcast day, shade on a clear day, and direct sunlight conditions, respectively. The Mid and High Coating treatments reflected about 9.2 and 23.4 more light than the Black samples. The lux values indicate that the titanium dioxide coating was effective in reflecting light radiation.

TABLE 5
Lux measurements by coating treatment at three light levels

| | Treatment | | | |
|---|---|---|---|---|
| Light level | Black | Mid | High | SE |
| Low, m² | 48^{a} | 505^{b} | 1,318^{c} | 37.3 |
| Medium, m² | 352^{a} | 3,366^{b} | 8,669^{c} | 216.9 |
| High, m² | 1,741^{a} | 15,978^{b} | 40,730^{c} | 990.5 |

^{a,b,c}Values within row without common superscripts differ (P < 0.001).

The model projected time to 50% of temperature increase, and the temperature at 10 min are shown in Table 6. The uncoated Black hide samples took over 3 min to reach 50% of the maximum temperature and reached about 70° C. in 10 min of exposure to the light source. The High coating treatment reached the 50% of maximum in 0.76 min, and at 10 min the temperature was only 31.7° C. The 38° C. greater (P<0.001) skin side temperature in the Black treatment than the High treatment indicates a greater reflectance of light energy from the surface of the coated samples and less energy absorbed.

TABLE 6
Heat transfer measurements by treatment

| | Treatment | | | |
|---|---|---|---|---|
| Item | Black | Mid | High | SE |
| Temperature @ | 70.1^{a} | 55.0^{b} | 31.7^{c} | 2.10 |
| 10 min, C. | | | | |
| Time to 50% | 3.25^{d} | 2.04^{e} | 0.76^{f} | 0.243 |
| temperature | | | | |
| increase, min | | | | |

^{a,b,c}Values within row without common superscripts differ (P < 0.001).
^{d,e,f}Values within row without common superscripts differ (P < .001).

The percentage of radiation reflected by the coating treatments over the wavelengths from 350 to 900 nm was determined. In the visible wavelength range (400 to 750 nm), the Black hides had 10 to 15% reflectance, the Mid coating treatment reflected 35 to 40% of the light, and the High coating treatment reflected 75 to 80% of the light energy.

In conclusion, the titanium dioxide pigment reflected the expected light energy when applied to hair-on cow hide rugs. Although the hides were not live animals, the results of this experiment show that coating cattle with a reflective pigment may be effective in helping to mitigate heat stress.

I claim:

1. A method of decreasing solar heat radiation absorption on an animal, comprising applying a highly infrared and solar reflective composition to the skin or hide of the animal.

2. The method of claim 1, wherein the animal is a bovine.

3. The method of claim 2, wherein the reflective composition is applied to at least a portion of the animal's back.

4. The method of claim 2, wherein at least a portion of the animal's hide to which the reflective composition is applied is black.

5. The method of claim 1, wherein the reflective composition comprises titanium dioxide or zinc oxide.

6. The method of claim 1, wherein the reflective composition comprises a water-releasing material capable of slowing releasing water therefrom to provide an evaporative cooling effect for the animal upon the animal's exposure to solar heat radiation.

7. A method of increasing solar heat radiation transfer to an animal by applying a highly infrared and thermally absorptive material to the skin or hide of the animal.

8. The method of claim 7, wherein the animal is a bovine.

9. The method of claim 8, wherein the absorptive material is applied to at least a portion of the animal's back.

10. The method of claim 8, wherein at least a portion of the animal's hide to which the reflective material is applied is white.

11. The method of claim 7, wherein the highly absorptive material comprises iron oxide or carbon black.

12. A non-toxic composition for use in managing the effects of solar radiation on an animal comprising:
from about 10% to about 50% by weight of one or more infrared radiation absorptive or reflective materials;
from about 1% to about 20% by weight of at least one adhesion-promoting or water resistance-enhancing materials; and
from about 20% to about 80% water.

13. The composition according to claim 12, wherein the composition comprises one or more infrared radiation absorptive materials, the one or more infrared radiation absorptive materials comprising iron oxide and/or carbon black.

14. The composition according to claim 12, wherein the composition comprises one or more infrared radiation reflective materials, the one or more infrared radiation reflective materials comprising titanium dioxide and/or zinc oxide.

15. The composition according to claim 12, wherein the composition further comprises one or more materials selected from the group consisting of thickeners, tackifiers, binders, humectants, waxes, acrylic polymers and copolymers, fragrances, insect repellants, and germicidal agents.

16. A method for reducing incidences of Buller-Steer Syndrome within cattle in a feedlot comprising applying to at least a portion of a population of cattle confined to a common pen a composition comprising a fragrance effective to mask the identification of one or more bullers within the common pen.
